SEQUENCE LISTING (3 sequences)

SEQ ID NO: 1 - DNA, 1269 bp, Coronavirus
  CDS (1)..(1269)
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 2 - protein, 422 aa, Coronavirus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - protein, 18 aa, Artificial Sequence
  A synthetic peptide sequence consisting of the amino acids 244-260 of SEQ ID NO2 and Cysteine
GQTVTKKSAAEASKKPRC

(19) United States
(12) Patent Application Publication
(10) Pub. No.: US 2008/0254440 A1
(43) Pub. Date: Oct. 16, 2008

(54) Anti-Sars Virus Antibody, Hybridoma Producing the Antibody and Immunoassay Reagent Using the Antibody

(76) Inventors: Yoshiaki Uchida, Tokyo (JP); Nobuyuki Fujii, Tokyo (JP); Yoshihiro Kurano, Tokyo (JP); Masahisa Okada, Tokyo (JP); Hiroyuki Kogaki, Tokyo (JP); Yasuji Kido, Tokyo (JP); Kazushige Miyake, Tokyo (JP)

Correspondence Address: BIRCH STEWART KOLASCH & BIRCH, PO BOX 747, FALLS CHURCH, VA, 22040-0747, US

(21) Appl. No.: 10/577,310

(22) PCT Filed: Oct. 29, 2004
(86) PCT No.: PCT/JP2004/016099

§ 371 Date: Feb. 22, 2007

(30) Foreign Application Priority Data
Oct. 31, 2003 (JP) 2003-373779
Feb. 10, 2004 (JP) 2004-034268

(51) Int. Cl. C12Q 1/70; C07K 16/18; C12M 1/00; C12N 5/06

(52) U.S. Cl. 435/5; 530/388.1; 435/339; 435/287.2

(57) ABSTRACT

A monoclonal antibody which specifically recognizes SARS virus is provided, and an immunoassay, immunoassay reagent and immunoassay device for detecting the SARS virus using the monoclonal antibody are disclosed. The monoclonal antibody according to the present invention is a monoclonal antibody against a nucleoprotein of a corona virus causing severe acute respiratory syndrome (SARS).

### TECHNICAL FIELD

The present invention relates to a monoclonal antibody against the capsid protein (hereinafter referred to as “nucleoprotein”) of the severe acute respiratory syndrome (SARS)-causing coronavirus, hybridoma which produces the monoclonal antibody, and to an immunoassay reagent and immunoassay device for SARS virus, which uses the monoclonal antibody as the immobilize antibody and/or labeled antibody.

### BACKGROUND ART

From 2002 to 2003, patients suffering from severe pneumonia were reported worldwide, and a number of death were reported in addition to the infected patients. The virus isolated from the patients was named SARS virus, and the virus was confirmed to be a new type of coronavirus. The whole genome has been sequenced by Michael Smith Genome Sciences Centre in British Columbia, Canada (Non-patent Literature 1).

After incubation period of 2 to 7 days from the infection by SARS virus, the SARS virus causes high fever higher than 38° C., coughs, headache, dyspnea and so on. Since the symptoms of SARS are similar to those of influenza, diagnosis whether the infection is by SARS or not at an early stage is demanded in order to select the appropriate treatments. Reported diagnoses of infection by SARS virus include the following:
1) Measurement of Antibody by ELISA: Antibodies (IgM/IgA) in sera of SARS patients may be detected after about 20 days from the manifestation of clinical symptoms.
2) Immunofluorescence Method: Immunofluorescence method using VERO cells infected with SARS virus (detecting IgM). Antibody in serum may be detected after about 10 days from the onset.
3) PCR Method: SARS virus genes from various specimens such as blood, feces and respiratory secretions are amplified and detected.
4) Cell Culture Method: Virus in a specimen from a SARS patient is infected to culture cells such as VERO cells and then detected.
Non-patent Literature 1: Science; 2003 May 30; 300(5624):1394-9

### DISCLOSURE OF THE INVENTION

### Problems Which the Invention Tries to Solve

Among the known methods for confirming infection by SARS virus, with the antibody test methods, the infection can be detected only after 10 days from the infection, and the highly reliable immunofluorescence method is complicated. As for the PCR method, since it is necessary to isolate and amplify a SARS-related gene, the method requires a special amplification apparatus and measurement apparatus, and is not a simple measurement method. As for the cell culture method, it is difficult to process a number of specimens at one time, and infection by SARS virus cannot be confirmed only by this method, even though the infection by coronavirus may be confirmed. An anti-SARS virus antibody having higher specificity and higher affinity is continuously demanded.

In view of the above-described circumstances, an object of the present invention is to provide a monoclonal antibody which specifically recognizes SARS virus, and to provide an immunoassay, immunoassay reagent and immunoassay device for detecting SARS virus.

### Means for Solving the Problems

The present inventors intensively studied for obtaining an anti-SARS virus monoclonal antibody having specificity to SARS virus and having a high affinity to obtain the desired monoclonal antibody by obtaining a nucleoprotein gene of SARS virus by polynucleotide synthesis utilizing PCR, preparing a transformant containing the gene using gene manipulation techniques, and immunizing, as an immunogen, the nucleoprotein of SARS virus obtained from the transformant. Further, the present inventors were able to develop an immunoassay reagent using the monoclonal antibody.

That is, the present invention provides an anti-SARS virus monoclonal antibody against nucleoprotein of a corona virus which causes severe acute respiratory syndrome (SARS), or an antigen-binding fragment thereof. The present invention also provides a hybridoma producing the monoclonal antibody according to the present invention, which hybridoma is obtained by fusing an anti-SARS virus monoclonal antibody-producing cell and a tumor cell. The present invention further provides a reagent for immunoassay of SARS-causing coronavirus, comprising the monoclonal antibody or the antigen-binding fragment thereof according to the present invention as at least one of immobilized antibody and labeled antibody. The present invention still further provides an immunoassay device comprising a detection zone having an anti-SARS virus antibody immobilized on a matrix through which liquid can be transported; and a labeled reagent zone on which a labeled anti-SARS antibody is spotted in such a manner that the labeled anti-SARS antibody is mobile; at least one of the antibody immobilized on the detection zone and the labeled anti-SARS virus antibody being the monoclonal antibody or the antigen-binding fragment thereof according to the present invention. The present invention still further provides an immunoassay of SARS virus, comprising detecting the SARS virus in a test sample by an immunoassay utilizing antigen-antibody reaction between the anti-SARS virus monoclonal antibody or the antigen-binding fragment thereof according to the present invention and the SARS virus in the test sample.

### EFFECTS OF THE INVENTION

Since the monoclonal antibody according to the present invention has a high specificity and high affinity to the nucleoprotein of SARS virus, the monoclonal antibody may be used for highly sensitive immunoassay of SARS virus. The hybridoma according to the present invention can provide a monoclonal antibody which specifically recognizes SARS virus. Further, the immunoassay reagent utilizing the monoclonal antibody according to the present invention may detect only the samples containing SARS virus or only the samples from SARS patients by simple operations.

### BRIEF DESCRIPTION OF THE DRAWINGS

FIG. 1 is a restriction map of a plasmid pW6A for expressing the nucleoprotein used as the immunogen, which was used in an Example of the present invention.

FIG. 2 schematically shows the results of SDS-polyacrylamide gel electrophoresis of a recombinant protein (S—N) expressed in an Example of the present invention.

FIG. 3 shows the results of Western blot indicating the reactivities of the monoclonal antibodies (rSN-18 antibody, rSN-122 antibody and rSN-150 antibody), which was carried out in an Example of the present invention.

FIG. 4 shows the results of Western blot indicating the reactivities of the monoclonal antibodies (rSN-21-2 antibody, rSN-29 antibody and rSN-122 antibody), which was carried out in an Example of the present invention.

FIG. 5 shows a schematic cross-sectional view of an embodiment of the immunoassay device for immunochromatography according to the present invention.

### BEST MODE FOR CARRYING OUT THE INVENTION

As described above, the monoclonal antibody according to the present invention is a monoclonal antibody against the nucleoprotein (that is, the capsid protein) (hereinafter referred to as simply “nucleoprotein”) of coronavirus causing SARS. The term “monoclonal antibody against the nucleoprotein” herein means a monoclonal antibody which undergoes antigen-antibody reaction with the nucleoprotein. Therefore, not only the monoclonal antibodies prepared by using the nucleoprotein as an immunogen, but also those prepared by using a partial region of the nucleoprotein or a variant of the partial region are within the scope of the present invention as long as they undergo antigen-antibody reaction with the nucleoprotein.

As is well-known, antibody fragments such as Fab fragment and F(ab′)₂ fragment, which have binding ability to the corresponding antigen (referred to as “antigen-binding fragment” in the present description) may be obtained by decomposing an antibody by papain or pepsin. The antigen-binding fragments of the monoclonal antibody according to the present invention may also be used in the same manner as the monoclonal antibody and are within the scope of the present invention.

The monoclonal antibody according to the present invention may be obtained by using the nucleoprotein as an immunogen. The amino acid sequence of the nucleoprotein is known (Non-patent Literature 1), and the amino acid sequence is shown in SEQ ID NO:2. The nucleotide sequence encoding the amino acid sequence shown in SEQ ID NO:2 is shown in SEQ ID NO:1. Thus, the monoclonal antibody according to the present invention may be obtained by using as an immunogen the polypeptide having the amino acid sequence shown in SEQ ID NO:2. A naturally occurring variant of the amino acid sequence shown in SEQ ID NO:2 may also be used. The nucleoprotein may not be necessarily highly purified, and crudely purified nucleoprotein may also be used as the immunogen. Proteins containing the amino acid sequence shown in SEQ ID NO:2 to which other amino acid sequence(s) is(are) attached to the N-terminal and/or C-terminal to the extent that the property as an immunogen is not adversely affected may also be used as the immunogen. Alternatively, the monoclonal antibody may also be obtained by using a partial region of the amino acid sequence shown in SEQ ID NO:2 as the immunogen. Such a partial region preferably comprises not less than 10 amino acids from the view point of specificity. Although the upper limit of the size of the partial region is less than the full length, even a peptide having 10 to 50 amino acids, preferably about 15 to 30 amino acids, may induce the monoclonal antibody according to the present invention. For example, in an Example below, it was confirmed that the monoclonal antibody according to the present invention may be obtained by using as an immunogen the peptide having the amino acid sequence shown in SEQ ID NO:3 (a sequence consisting of 244th to 260th amino acids in SEQ ID NO:2 and one cystein). Such a relatively small peptide may be easily synthesized chemically using a commercially available peptide synthesizer, which is convenient. The antigenicity of such a relatively short peptide may be increased by conjugating the peptide with a carrier protein such as keyhole limpet hemocyanin (KLH) or bovine serum albumin (BSA) to constitute an immunogen.

Although it is preferred to use as the immunogen the nucleoprotein having the amino acid sequence shown in SEQ ID NO:2 or a partial region thereof, especially the full length of the nucleoprotein, the monoclonal antibody according to the present invention may also be obtained by using as an immunogen a polypeptide having the same amino acid sequence shown in SEQ ID NO:2 except that a small number of amino acid(s) in the amino acid sequence shown in SEQ ID NO:2 or a partial region thereof is(are) substituted and/or deleted, and/or a small number of amino acid(s) is(are) inserted thereinto. The amino acid sequence of such an immunogen preferably has an identity as high as possible to the amino acid sequence shown in SEQ ID NO:2 or a partial region thereof. The sequence identity may preferably be not less than 90%, more preferably not less than 95%. The identity between amino acid sequences may easily be calculated by using a well-known computer software such as BLAST, and such a software is opened for public use in the internet. In cases where a small number of amino acid(s) is(are) substituted, deleted and/or inserted, the total number of amino acid(s) which is(are) substituted, deleted and/or inserted may preferably be one to several. The 20 types of amino acids constituting the naturally occurring proteins may be classified into groups each of which has similar properties, for example, into neutral amino acids with side chains having low polarity (Gly, Ile, Val, Leu, Ala, Met, Pro), neutral amino acids having hydrophilic side chains (Asn, Gln, Thr, Ser, Tyr, Cys), acidic amino acids (Asp, Glu), basic amino acids (Arg, Lys, His) and aromatic amino acids (Phe, Tyr, Trp). In most cases, substitutions of amino acids within the same group do not substantially change the immunogenic properties of the immunogen.

The nucleoprotein of SARS virus used as the above-described immunogen may be obtained by, for example, the following method using gene manipulation technique:

By amplifying the gene region (SEQ ID NO:1) encoding the nucleoprotein by PCR, a DNA fragment encoding a polypeptide substantially containing the amino acid sequence shown in SEQ ID NO:2 is obtained. That is, for example, RNA is extracted from SARS virus, and RT-PCR is performed. That is, the region from the 5′-end of the N protein gene to the NheI restriction site (a restriction site such as EcoRI site is added to the 5′-end), and the region from the NheI site to the 3′-end of the N protein (a restriction site such as BamHI site is added to the 3′-end) are amplified by RT-PCR, respectively. By treating each of the fragments with a restriction enzyme and ligating the resulting fragments, a DNA fragment encoding a polypeptide substantially containing the amino acid sequence shown in SEQ ID NO:2 may be obtained. By inserting this DNA fragment into an appropriate expression vector, an expression vector may be constructed. In another method, a DNA fragment encoding a polypeptide substantially containing the polypeptide having the amino acid sequence shown in SEQ ID NO:2 or 3 may be chemically synthesized based on the above-described nucleotide sequence. The thus obtained DNA fragment is incorporated into an expression vector having an appropriate marker gene such as ampicillin-resistant gene, and a host such as E. coli is transformed with the resulting vector to obtain a transformant. By culturing the obtained transformant and by purification of the culture medium, the above-described nucleoprotein of SARS virus may be obtained. Polypeptides such as those containing the sequence shown in SEQ ID NO:3 may also be obtained by a known synthesis method using a chemical synthesizer.

The above-described anti-SARS virus monoclonal antibody may be produced by a hybridoma obtained by immunizing an animal with the above-described immunogen, and fusing anti-nucleoprotein antibody-producing cells obtained from the animal and tumor cells.

The above-described hybridoma may be obtained by, for example, the following method: That is, the nucleoprotein obtained as described above as an immunogen is intrapectoneally or intravenously administered to an animal such as mouse together with Freund's complete adjuvant, dividedly in twice or three times, at 2 to 3-week intervals. Then the antigen-producing cells originated from the spleen or the like obtained from the immunized animal and tumor cells which can proliferate in vitro such as myeloma cells selected from immortalized cell lines such as myeloma cell line are fused.

As the above-described fusion method, the polyethylene glycol method according to the conventional method by Kohler and Milstein (Nature, Vol. 256, page 495, 1975), as well as the Sendai virus method may be employed.

Selection of hybridomas producing the antibody which recognizes the nucleoprotein of SARS virus from the fused cells may be attained by, for example, the following method: That is, cells which are alive in HAT medium are selected as hybridomas from the fused cells. Then the culture medium of each of the obtained hybridomas is reacted with highly purified nucleoprotein of SARS virus immobilized on an assay plate. Then the assay plate is reacted with anti-mouse immunoglobulin (Ig) or the like. By such an EIA, hybridomas producing monoclonal antibodies which specifically recognize the nucleoprotein of SARS virus may be selected.

The hybridoma according to the present invention is not restricted as long as it produces a monoclonal antibody which specifically recognizes the nucleoprotein. Examples of the hybridoma include the 6 hybridomas established by the above-described method by the present inventors.

The 6 hybridomas were named hybridoma rSN-18, hybridoma rSN-122, hybridoma rSN-150, hybridoma rSN-21-2, hybridoma rSN-29 and hybridoma SN5-25, respectively. These hybridomas have been deposited with International Patent Organism Depositary, National Institute of Advanced Industrial Science and Technology (address: AIST Tsukuba Central 6, 1-1-1 Higashi, Tsukuba, Ibaraki, Japan). That is, hybridoma rSN-18 has been deposited under Accession No. FERM P-19572 (date of receipt: Oct. 24, 2003), hybridoma rSN-122 has been deposited under Accession No. FERM P-19573 (date of receipt: Oct. 24, 2003), hybridoma rSN-150 has been deposited under Accession No. FERM P-19574 (date of receipt: Oct. 24, 2003), hybridoma rSN-21-2 has been deposited under Accession No. FERM P-19619 (date of receipt: Dec. 26, 2003) and hybridoma rSN-29 has been deposited under Accession No. FERM P-19620 (date of receipt: Dec. 26, 2003). These depositions were converted to international depositions with International Patent Organism Depositary, National Institute of Advanced Industrial Science and Technology (address: AIST Tsukuba Central 6, 1-1-1 Higashi, Tsukuba, Ibaraki, Japan) on Oct. 18, 2004. That is, the Accession No. of hybridoma rSN-18 was converted to FERM BP-10143, the Accession No. of hybridoma rSN-122 was converted to FERM BP-10144, the Accession No. of hybridoma rSN-150 was converted to FERM BP-10145, the Accession No. of hybridoma rSN-21-2 was converted to FERM BP-10146, and the Accession No. of hybridoma rSN-29 was converted to FERM BP-10147, respectively.

Each of the above-described hybridoma may be cultured in a culture medium ordinarily used for cell culture. The monoclonal antibody may be recovered from the culture supernatant. The monoclonal antibody may also be obtained by transplanting the hybridomas into the abdominal cavity of an animal of the same species as the animal from which the hybridomas were derived, recovering ascites after proliferation of the hybridomas, and by purifying the ascites.

The monoclonal antibody may be recovered by a purification method conventionally employed. Examples of the purification method include gel permeation chromatography, ion-exchange chromatography and affinity chromatography using protein A.

The reactivity of the monoclonal antibody may be confirmed by a usual method. The reactivity of the monoclonal antibody of the present invention is confirmed by using as an index the specificity of the reaction with the nucleoprotein of SARS virus.

The monoclonal antibody according to the present invention may be used for immunoassays for the detection or quantification of SARS virus. The immunoassays per se are well-known and any of the well-known immunoassays may be employed. That is, classifying the known immunoassays according to the reaction type, known immunoassays include sandwich immunoassays, competition immunoassays, agglutination immunoassays, Western blot and the like. Classifying the known immunoassays according to the label employed, known immunoassays include fluorescence immunoassays, enzyme immunoassays, radio immunoassays, biotin immunoassays and the like. Any of these immunoassays may be employed. Further, diagnosis may be attained by immunohistostaining. In cases where a labeled antibody is used in the immunoassay, the methods per se for labeling an antibody are well-known, and any of the well-known methods may be employed.

These immunoassays per se are well-known in the art, and so it is not necessary to explain these immunoassays in the present specification. Briefly, in sandwich immunoassays, for example, the antibody of the present invention or an antigen-binding fragment thereof is immobilized on a solid phase as a first antibody. The first antibody is then reacted with a sample, and after washing the solid phase, the resultant is then reacted with a second antibody which reacts with the enzyme of the present invention by antigen-antibody reaction. After washing the solid phase, the second antibody bound to the solid phase is measured. By labeling the second antibody with an enzyme, fluorescent substance, radioactive substance, biotin or the like, measurement of the second antibody bound to the solid phase may be attained by measuring the label. The above-mentioned measurement is conducted for a plurality of standard samples each containing a known concentration of the enzyme, and the relationship between the concentrations of the enzyme in the standard samples and the measured amounts of the label is plotted to prepare a calibration curve. The enzyme in a test sample may be quantified by applying the measured amount to the calibration curve. It should be noted that the above-mentioned first antibody and the above-mentioned second antibody may be exchanged. In agglutination immunoassays, the antibody according to the present invention or an antigen-binding fragment thereof is immobilized on particles such as latex particles, and the particles are reacted with a sample, followed by measurement of the absorbance. The above-mentioned measurement is conducted for a plurality of standard samples each containing a known concentration of the enzyme, and the relationship between the concentrations of the enzyme in the standard samples and the measured absorbance is plotted to prepare a calibration curve. The enzyme in a test sample may be determined by applying the measured absorbance to the calibration curve.

The sample to be subjected to the above-described immunoassay is not restricted as long as it contains the nucleoprotein of SARS virus, and examples of the sample include extracts of body fluids such as nasal swab, nasal aspirate or pharyngeal swab, respiratory secretion, cell or tissue homogenates and the like, as well as serum, plasma and whole blood, collected from human or animal.

By using the above-described monoclonal antibody according to the present invention as at least one of the solid phase antibody and labeled antibody, a reagent for measuring SARS virus may be produced. As the solid phase on which the above-described monoclonal antibody is immobilized, various solid phases used in conventional immunoassays may be used. Examples of such solid phases include various solid phases such as ELISA plates, latices, gelatin particles, magnetic particles, polystyrenes and glasses, insoluble carriers such as beads and matrices through which liquid can be transported and the like. The labeled antibody may be produced by labeling an antibody with an enzyme, colloidal metal particle, colored latex particle, luminescent substance, fluorescent substance, radioactive substance or the like. By combining reagents such as these solid phase antibodies and/or labeled antibodies, reagents used in enzyme immunoassays, radioimmunoassays, fluoroimmunoassays or the like may be produced. These measurement reagents are the reagents for measuring an antigen of interest present in the test sample by sandwich immunoassay or competitive binding immunoassay. The immunoassay device of the present invention for measuring SARS virus utilizes the principle of immunochromatography. The device comprises a detection zone having the monoclonal antibody of the present invention immobilized on a matrix through which liquid can be transported, and a labeled reagent zone having a labeled anti-SARS virus monoclonal antibody of the present invention spotted movably on the above-described matrix.

A reagent for the above-described sandwich immunoassay may be provided by, for example, providing two monoclonal antibodies according to the present invention, and using one of them as the above-described labeled antibody and using the other as the immobilized antibody bound to the solid phase. First, the solid phase antibody is reacted with a sample containing an antigen to be measured, and then the antigen bound to the solid phase antibody is reacted with the labeled antibody (second antibody). By detecting the presence of the label bound to an insoluble carrier, immunoassay may be attained. Similarly, immunoassay may be carried out by reacting the solid phase antibody with a sample containing an antigen to be measured, and then reacting the antigen bound to the solid phase antibody with the labeled antibody (second antibody), followed by measuring the amount of the label, that is, the labeled antibody, bound to the insoluble carrier. As reagents for immunoassay used in sandwich immunoassay, although one type of monoclonal antibody may be used as both of the solid phase antibody and the labeled antibody (for example, in cases where the antigen is multimeric), it is usually preferred to use two or more types of antibodies recognizing two different epitopes contained in the antigen to be measured. That is, it is preferred to select a solid phase antibody and a labeled antibody respectively from monoclonal antibodies each of which recognizes a different epitope. Further, as the solid phase antibody and as the labeled antibody, respectively, a plurality of monoclonal antibodies selected from two or more types of monoclonal antibodies may be used in combination.

As a reagent for immunoassay used in competitive binding immunoassay, for example, a certain amount of a virus antigen labeled with an enzyme, colloidal metal particle, colored latex particle, luminescent substance, fluorescent substance, radioactive substance or the like is prepared. Using this reagent, for example, a certain amount of the monoclonal antibody of the present invention, the above-described labeled virus antigen and a sample containing the antigen to be measured may be reacted competitively, and the amount of the antigen to be measured may be determined based on the amount of the labeled virus antigen bound or not bound to the antibody, thereby attaining immunoassay.

In the present invention, a method such as physical adsorption or chemical bond may be used for binding the above-described antibody or antigen to the solid phase or the label (see “PROTEIN, NUCLEIC ACID AND ENZYME”, Extra Edition, vol. 31, pp. 37-45 (1987)).

By utilizing the anti-SARS virus monoclonal antibody of the present invention for an immunoassay device which utilizes the principle of immunochromatography, SARS virus present in a sample may be detected easily without using a special measuring apparatus. This immunoassay device comprises a belt-like matrix through which liquid can be transported (developed) by capillary action, which matrix comprises a SARS virus detection zone on which at least one type of anti-SARS virus monoclonal antibody is immobilized (solid-phased), a labeled reagent zone on which a labeled anti-SARS virus monoclonal antibody is spotted movably; a sample-spotting zone; a developer-supply zone having a developer pad mounted at one end of the above-described matrix in the longitudinal direction; and a developer-absorption zone formed at the other end of the above-described matrix in the longitudinal direction.

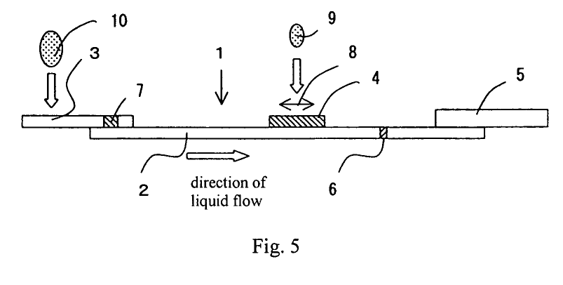

A schematic cross-sectional view of a preferred embodiment of such an immunoassay device for immunochromatography is shown in FIG. 5. In FIG. 5, reference numeral 1 denotes an immunoassay device for immunochromatography, reference numeral 2 denotes a matrix through which liquid can be transported, reference numeral 3 denotes a developer-supply zone having a dried substrate zone 7, reference numeral 4 denotes a labeled reagent zone, reference numeral 5 denotes a developer-absorption zone, reference numeral 6 denotes a detection zone, reference numeral 8 denotes a sample-spotting zone, reference numeral 9 denotes a sample and reference numeral 10 denotes a developer. The constituents of this immunoassay device will now be described.

### Matrix

The matrix in this immunoassay device is made of a belt-shaped, water-absorptive material in which liquid can be transported by capillary action. Examples of the water-absorptive material include cellulose and derivatives thereof such as cellulose and nitrocellulose, and filter papers made of glass fibers alone or containing glass fibers, membranes and porous materials. Although the size of the matrix is not restricted, those in the form of strip having a width of about 3 mm to 10 mm, and a length of about 30 mm to 100 mm are preferred because they have good ease of handling. The thickness of the matrix may be, for example, 100 μm to 1 mm. To prevent non-specific adsorption of the proteins originated from the sample to the matrix during the measurement, a part or the entire matrix may be blocked with an animal serum protein such as bovine serum albumin (BSA), casein, sucrose or the like.

### Detection Zone

In the detection zone, a SARS virus-detection section in which the anti-SARS virus monoclonal antibody is immoblized on the matrix may be provided. The anti-SARS virus monoclonal antibody in the detection section is preferably arranged on the matrix, in the form of a line perpendicular to the direction of the flow of the liquid (longitudinal direction of the matrix) for attaining the measurement with high sensitivity.

The anti-SARS virus monoclonal antibody in the detection zone is the above-described antibody, and the monoclonal antibody may be used individually or a plurality of the antibodies may be used in combination. The anti-SARS virus monoclonal antibody may be IgG antibody or IgM antibody, or may be Fab, Fab′, F(ab′)₂ or the like which is an fragment of these antibodies.

The anti-SARS virus antibody immobilized in the detection section may be physically adsorbed directly in the detection zone on the matrix, or may be fixed by chemical bond such as covalent bond. Alternatively, the anti-SARS virus monoclonal antibody may be bound to a water-insoluble carrier and the carrier may be incorporated in the matrix. Examples of the insoluble carriers include the particles obtained by insolubilizing a mixture of gelatin, gum arabic and sodium hexametaphosphate (Japanese Patent Publication (Kokai) No. 63-29223), polystyrene latex particles, glass fibers and the like. The anti-SARS virus monoclonal antibody may be bound to the insoluble carrier by the above-described chemical bond or by physical adsorption.

On the matrix, the detection section is formed downstream of the labeled reagent zone, the sample-spotting zone and the developer-supply zone in the direction of the flow of the developer, and located upstream of the developer-absorption zone. The detection section may be arranged on the matrix in the form of a line having a width of about 0.5 mm to 5 mm, or in the form of a plurality of lines. In the case of a matrix having a width of about 5 mm, the detection section may be formed by spotting the above-described antibody and/or antigen usually in an amount of about 0.1 μg to 10 μg, respectively, and drying the matrix.

### Labeled Reagent Zone

The labeled reagent zone may be formed by spotting a labeled anti-SARS virus monoclonal antibody movably. This labeled reagent zone may be formed upstream of the above-described detection zone in the direction of the flow of the developer from the developer-supply zone. This labeled reagent zone may be formed by spotting the labeled reagent on the matrix, by laminating a water-absorptive pad containing the labeled reagent, or by incorporating the labeled reagent in a part or the entire region of the matrix which intimately contacts the pad. As the water-absorptive pad, the same pad as used for the sample-spotting zone hereinbelow described may be used.

At least one of the labeled antibody and the antibody immobilized in the detection zone is the anti-SARS virus monoclonal antibody according to the present invention, and both of them are preferably the anti-SARS virus monoclonal antibody according to the present invention. As the labeled anti-SARS virus monoclonal antibody, fragments thereof may be employed as in the case of the above-described antibody in the detection zone.

The labeled anti-SARS virus monoclonal antibody may be prepared by binding the anti-SARS virus monoclonal antibody with the label. Examples of the label include enzymes, colloidal metal particles, colored latex particles, fluorescent latex particles, luminescent substances, fluorescent substances and the like. As the enzyme, various enzymes used in enzyme immunoassays (EIA) may be employed. Examples of the enzyme include alkaline phosphatase, peroxidase, β-D-galactosidase and the like. Examples of the colloidal metal particles include colloidal gold particles, colloidal selenium particles and the like.

The known methods using covalent bond or non-covalent bond may be used for binding the label and the anti-SARS virus monoclonal antibody. Examples of the binding method include glutaraldehyde method, periodate method, maleimide method, pyridyl disulfide method and methods using various cross-linking agents (see, for example, “PROTEIN, NUCLEIC ACID AND ENZYME”, Extra Edition, vol. 31, pp. 37-45 (1985)). In the methods using a cross-linking agent, for example, N-succinimidyl-4-maleimide butyric acid (GMBS), N-succinimidyl-6-maleimide hexanoic acid, N-succinimidyl-4-(N-maleimidomethyl)cyclohexane-1-carboxylic acid or the like may be used as a cross-linking agent. In the methods using covalent bond, the functional groups existing in the antibody may be used. Alternatively, the labeled anti-SARS virus monoclonal antibody may be prepared by binding the label to the functional group using the above-described binding method after introducing a functional group such as thiol group, amino group, carboxyl group, hydroxyl group or the like by a conventional method. In the methods using non-covalent bond, the physical adsorption or the like may be used.

Although the amount of the labeled anti-SARS virus monoclonal antibody may be appropriately selected depending on the expected amount of the test substance in the sample, it is usually about 0.01 μg to 5 μg in terms of dry weight. The labeled anti-SARS monoclonal antibody may be applied together with a stabilizer, solubilization-adjusting agent or the like.

### Sample-Spotting Zone

The sample-spotting zone may be formed in the matrix at a site downstream of the developer-supply zone and upstream of the detection zone in the direction of the flow of the developer, without incorporating a reagent or the like. The sample-spotting zone may also be formed at 1) a prescribed site downstream of the developer-supply zone and upstream of the labeled-reagent zone in the direction of the flow of the developer, 2) a prescribed site downstream of the labeled reagent zone and upstream of the detection zone in the direction of the flow of the developer, or 3) in a prescribed site on the labeled reagent zone. In the device in which the sample-spotting zone is formed in the labeled reagent zone, it is preferred to add the water-absorptive pad containing the labeled reagent for carrying out the assay efficiently as mentioned above. By using the device comprising the pad, since a large amount of sample fluid may be spotted, a minor component in the sample may be measured with high detection sensitivity. The material constituting the water-absorptive pad is selected from the materials which scarcely adsorb the labeled reagent and the SARS virus in the sample. Examples of such materials include porous materials made of synthetic or natural macromolecular compounds such as polyvinyl alcohol (PVA), non-woven fabric, cellulose and the like, and these materials may be employed individually or in combination. Although the size, thickness, density and the like of the pad are not restricted, it is usually preferred to use a pad having longitudinal and lateral lengths of about 3 mm to 10 mm, and a thickness of about 0.5 mm to 4 mm for carrying out the assay efficiently.

### Developer-Supply Zone

The developer-supply zone is the zone formed at one end of the matrix in the longitudinal direction, to which the developer is supplied. The assay may be started by immersing this zone in the developer contained in a vessel in an amount at least sufficient to reach the developer-absorption zone. A liquid bath containing the developer may be attached to the developer-supply zone, and the assay may be started by breaking a cover of the liquid bath thereby bringing the developer into contact with the matrix. The developer may appropriately contain a surfactant, buffering agent, stabilizer, antibacterial agent or the like. In cases where an enzyme is used as the label, the substrate may be added to the developer in addition to the substrate zone hereinbelow described. Examples of the buffer solution containing a buffer agent include acetate buffer, borate buffer, Tris-HCl buffer, diethanolamine buffer and the like. On the developer-supply zone, a developer pad may be mounted to stably and continuously supply the developer to the matrix. As the developer pad, a filter paper made of, for example, cellulose or cellulose derivative may be employed.

### Developer-Absorption Zone

The developer-absorption zone is provided in the matrix at the end other than the end at which the above-described developer-supply zone is provided. This zone is provided for absorbing the developer supplied to the matrix so as to fluently carry out the assay. The developer-absorption zone may be provided by elongating the matrix. Alternatively, the absorption zone may be provided by arranging a water-absorptive material on the matrix. In this case, the development of the developer may be accelerated. As the water-absorptive material, filter papers having a high water holding capacity, made of natural macromolecular compounds, synthetic macromolecular compounds or the like, or sponge or the like may be employed. It is preferred to arrange in the developer-absorption zone an absorptive material in the form of a pad having a volume enough to absorb the whole developer. In cases where the developer-absorption zone is provided by laminating the absorptive material on or below the matrix, a compact immunoassay device may be produced.

### Substrate Reagent Zone

In cases where an enzyme is used as the label contained in the labeled reagent zone, the substrate may be contained in the developer as mentioned above, or a substrate reagent zone may be provided in the matrix in the vicinity of the developer-supply zone. The substrate reagent zone may preferably be formed in the above-described developer pad mounted on the developer-supply zone by incorporating the substrate in the developer pad for increasing the amount of the substrate so as to carry out the assay with high sensitivity.

As the substrate, various coloring substrates, fluorescent substrates, luminescent substrates or the like described below may be used depending on the enzyme in the labeled reagent.

### (a) Coloring Substrates

For Peroxidase: 2,2′-azino-bis(3-ethylbenzothiazolin-6-sulfonate) (ABTS), 3,3′,5,5′-tetramethylbenzidine (TMB) and diaminobenzidine (DAB), each in combination with hydrogen peroxide;

For Alkaline Phosphatase: 5-bromo-4-chloro-3-indolyl phosphate (BCIP), p-nitrophenyl phosphate (p-NPP) and 5-bromo-4-chloro-3-indolyl phosphate disodium salt (BCIP.Na)

### (b) Fluorescent Substrates

For Alkaline Phosphatase: 4-methylumbelliferyl phosphate (4MUP)

For β-D-galactosidase: 4-methylumbelliferyl-β-D-galactoside (4MUG)

### (c) Luminescent Substrates

For Alkaline Phosphatase: 3-(2′-spiroadamantan)-4-methoxy-4-(3″-phosphoryloxy)phenyl-1,2-dioxetane•2 sodium salt (AMPPD)

For β-D-galactosidase: 3-(2′-spiroadamantan)-4-methoxy-4-(3″-β-D-galactopyranosyl)phenyl-1,2-dioxetane (AMGPD)

For Peroxidase: luminol and isoluminol, each in combination with hydrogen peroxide.

The substrate zone may usually be formed by applying an aqueous solution of the substrate in the form of a line on the developer pad, and drying the solution. If desired, a signal-increasing agent, stabilizer, solubilization-adjusting agent or the like may be added. The site of the substrate zone is not restricted as long as it is within the developer pad mounted on the end of the matrix. The amount of the substrate added to the developer or the developer pad may be selected depending on the assay conditions, and may usually be about 5 μg to 500 μg per device.

Method for using Immunoassay Reagent

With the immunoassay reagent according to the present invention, SARS virus in various samples may be assayed. The assay may be carried out by first supplying a sample to the sample-spotting zone of the immunoassay device of the present invention, and then supplying the developer to the developer pad, thereby developing the sample in the matrix. The developer moves in the matrix by capillary action to reach the developer-absorption zone. The components in the sample, which have not been bound to the detection zone, the enzyme-labeled reagent and the like, are absorbed by the developer-absorption zone, and the development is completed. After a prescribed time (usually 10 minutes to 20 minutes), the detection zone is observed, and the label bound to the detection section by the SARS virus antigen in the sample is detected and/or measured, thereby measuring the SARS virus. The detection may be carried out by visual observation or by using a measuring device such as colorimeter, fluorophotometer, photon counter, photosensitive film or the like, depending on the label. For the measurement, the method in which the coloring of the detection zone is visually observed, for example, is simple. By this method, by using a color chart corresponding to the concentration of the SARS virus, a semiquantitative assay may be attained. Quantification may also be attained by digitizing the coloring of the detection zone by a colorimeter or the like.

The matrix may be laminated and fixed on a support made of a plastic, metal, paper or the like. By fixing the matrix in a case made of a plastic or the like, providing a bath containing the developer in the developer-supply zone, and covering the matrix with a case having through holes at least at the sites of sample-spotting zone and detection zone, a device having a good ease of handling may be constituted.

As the sample to be assayed by the above-described reagent is not restricted as long as it contains the nucleoprotein of SARS virus. Examples of the samples include sera, plasma and whole blood from human and animals; body fluid extracts such as nasal swab, nasal aspirate and throat swab; respiratory secretion, cell homogenates and tissue homogenates. These samples in the form of solutions containing SARS virus may be used as they are. Alternatively, solutions containing the virus treated with a surfactant such as nonionic surfactant, anionic surfactant or the like may be used. Examples of the nonionic surfactant include Nonidet (Nonidet T-40), Triton and Brij; and examples of anionic surfactant includes SDS.

The nucleoprotein of SARS virus distributed in cells, tissues or the like may also be directly measured by fixing the various cells, tissues and the like originated from human or animals, and reacting the monoclonal antibody according to the present invention therewith. Further, the so called Western blotting, affinity chromatography or the like may be carried out using the monoclonal antibody according to the present invention.

By applying the measurement method of the nucleoprotein of SARS virus using the monoclonal antibody according to the present invention to various samples from human or animals, diagnosis of infection by SARS virus may be carried out. By using the monoclonal antibody according to the present invention, the nucleoprotein of SARS virus in various body fluids, cells, tissues and the like from human or animals may be directly measured by immunochemical or immunohistochemical method. It is suspected that SARS virus infected from mammals, birds or the like to human. Thus, in addition to the measurement of human samples, by measuring animal samples, the monoclonal antibody according to the present invention may also be used for the clarification of infection route.

In the above description of immunoassay, reagent and device, an antigen-binding fragment of the monoclonal antibody according to the present invention may be used in place of the monoclonal antibody of the present invention.

### EXAMPLES

The present invention will now be described by way of Reference Examples and Examples. However, the present invention is not restricted to the following Examples.

### Reference Example 1

### Construction of Plasmid

Full length nucleoprotein (referred to as “N protein”) gene consists of 1270 base pairs. Based on the reported gene sequence, the N protein gene was divided into two fragments at the restriction site of NheI which hydrolyzes the N protein gene at about the center thereof. Oligomers of 50 to 55 bases having an overlapping of 15 bases each other were annealed with the gene and the resulting product was subjected to extension reaction under conditions for synthesizing DNA, followed by amplification by PCR. This operation was repeated. The PCR was performed using, for the former half fragment, a forward primer having an EcoRI site at its 5′-end region and a reverse primer having an NheI site at its 3′-end region, and, for the latter half fragment, a reverse primer having a BamHI site at its 3′-end region and a reverse primer having an NheI site at its 5′-end region.

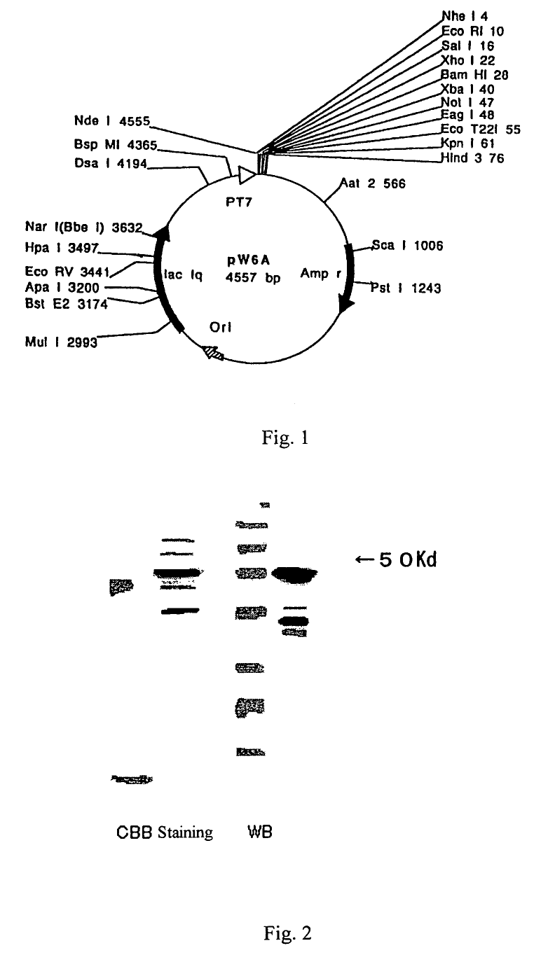

After purifying the resulting fragments by PCR Purification Kit from QIAGEN, the former half fragment was hydrolyzed with EcoRI and NheI, and the latter half fragment was hydrolyzed with NheI and BamHI. The obtained fragments were inserted into the EcoRI-BamHI site of an expression plasmid pW6A shown in FIG. 1 to prepare a plasmid pWS—N. E. coli BL21 (DE3) (obtained from Brookhaven National Laboratory) was transformed with the obtained plasmid to obtain an ampicillin-resistant transformant E. coli BL21/(DE3)/pWS—N. The nucleotide sequence and amino acid sequence of the nucleoprotein are shown in SEQ ID NOs:1 and 2, respectively.

### Reference Example 2

### Expression of Recombinant Protein (S—N)

The transformant prepared in Reference Example 1 was cultured in 2 ml of LB medium containing 50 μg/ml of ampicillin at 37° C. After growing the transformant in a preliminary culture until the OD of the culture medium reached to 0.6 to 0.8, IPTG was added to a concentration of 0.4 mM to induce the expression, and the culture was continued for another 3 hours. After recovering the bacterial cells by centrifugation of 1.5 ml of culture medium at 5000 rpm for 2 minutes, the cells were suspended in 100 μl of buffer (10 mM Tris-HCl, pH 8.0, 0.1 M sodium chloride, 1 mM EDTA), and the suspension was subjected to sonication to completely disrupt the cells. The thus obtained product was used as the bacterial cell sample.

To 8 μl of the bacterial cell sample, 4 μl of 3×SDS polyacrylamide buffer (0.15 M Tris-HCl, pH 6.8, 6% SDS, 24% glycerol, 6 mM EDTA, 2% 2-mercaptoethanol, 0.03% bromphenol blue) was added, and the resulting mixture was subjected to SDS-polyacrylamide gel electrophoresis. After the electrophoresis, the developed sample was transferred to a nitrocellulose filter, and the filter was subjected to blocking with 1% BSA, followed by reacting the resulting filter with anti-N5 peptide serum 1000-fold diluted with phosphate buffer (10 mM phosphoric acid, pH 7.4, containing 0.15 M sodium chloride). The resulting filter was then reacted with peroxidase-labeled anti-mouse Ig rabbit polyclonal antibody (produced by DAKO), and, after washing, 10 ml of substrate coloring solution (0.01% aqueous hydrogen peroxide solution, 0.6 mg/ml 4-chloro-1-naphthol) was added, thereby coloring the filter. The results are shown in FIG. 2.

The anti-N5 peptide serum was separated from the blood recovered from a mouse immunized with the N5 peptide-KLH conjugate prepared as described in Reference Example 4.

### Reference Example 3

### Purification of Soluble S—N

The E. coli BL21(DE3)/pWS—N prepared in Reference Example 1 was cultured in LB medium containing ampicillin at 37° C. The transformant was grown in a preliminary culture until a cell population in terms of OD at 600 nm reached to about 0.7, and IPTG was added to 0.4 mM, thereby inducing the expression. After culturing for 18 hours, E. coli was recovered by centrifugation. To the recovered E. coli, 20 mM Tris-HCl, pH 8.0, 1 mM PMSF (phenylmethylsulfonyl fluoride) was added, and the resulting mixture was sonicated while cooling the mixture on ice. After the centrifugation, ammonium sulfate was added to the soluble fraction S—N, and 20-40% fraction was recovered. This ammonium sulfate fraction was applied to SP Sepharose First Flow (produced by AMERSHAM) equilibrated with 20 mM phosphate buffer, pH 6.9, containing 0.1 M sodium chloride and 8M urea, and eluted with 20 mM phosphate buffer, pH 6.9, containing 0.2 M sodium chloride and 8M urea, thereby carrying out purification. The eluted fraction was dialyzed against 20 mM Tris-HCl buffer, pH 8.0, containing 0.2M sodium chloride. The obtained product was subjected to SDS-polyacrylamide gel electrophoresis and Western blot as in Reference Example 2, thereby confirming the degree of purification. As a result, a single band was shown.

### Example 1

### Establishment of Anti-N Protein Monoclonal Antibodies

Anti-N protein monoclonal antibodies were prepared by immunizing mice with the recombinant N protein prepared in Reference Example 3, and fusing the lymphocytes from the spleen of the mice and myeloma cells. That is, BALB/C mice were first immunized with the recombinant N protein emulsified with Freund's complete adjuvant in an amount of 50 to 100 μg/mouse, and 2 to 3 weeks later, second immunization was performed with the same antigen emulsified with Freund's incomplete adjuvant in an amount of 50 to 100 μg/mouse. The antibody titer was checked by solid phase ELISA using a 96-well ELISA plate coated with the recombinant N protein. To the mice in which the raise of the antibody titer was observed, free recombinant N protein was intravenously administered in an amount of 25 to 100 μg. Three to four days later, spleen was removed from each mouse and spleen cells were separated. The obtained spleen cells were mixed with mouse myeloma cells (P3U1) preliminarily cultured in RPMI-1640 medium at a mixing ratio of 1:2 to 1:5, and cell fusion was performed using PEG (produced by Boehringer). The fused cells were suspended in HAT medium and dividedly applied to a 96-well culture plate, followed by incubation at 37° C. in a CO₂ incubator.

The screening was carried out by the above-described solid phase ELISA. More particularly, a solution of the recombinant N protein with a concentration of 1 μg/ml was added to a 96-well ELISA plate (produced by PHARMACIA) in an amount of 50 μl/well, and the plate was left to stand overnight at 4° C., thereby adsorbing the recombinant N protein to the wells. Each well was blocked with 1% skim milk and washed three times with washing buffer (PBS containing 0.05% Tween 20). To each well, 50 μl of the supernatant of the culture medium in which cell fusion was performed was added, and the resultant was allowed to react at 37° C. for 1 hour. Each well was then washed 3 times with the washing buffer in the same manner as described above, and POD-labeled anti-mouse immunoglobulin antibody (produced by DAKO) was added, followed by allowing the mixture to react at 37° C. for 1 hour. After washing the wells 4 times with the washing buffer, the substrate ABTS was added, and the wells which colored were selected. The cells in the selected wells were transferred to a 24-well culture plate and cultured in a CO₂ incubator at 37° C., and the cells were cloned by the limiting dilution method to establish 5 hybridomas which produce the anti-N protein monoclonal antibodies described below, that is, hybridomas rSN-18, rSN-122, rSN-150, rSN-21-2 and rSN-29. These hybridomas have been deposited with the above-described International Patent Organism Depositary under the Accession Nos. FERM P-19572, FERM P-19573, FERM P-19574, FERM P-19619 and FERM P-19620, respectively. These depositions were converted to international depositions with International Patent Organism Depositary, National Institute of Advanced Industrial Science and Technology (address: AIST Tsukuba Central 6, 1-1-1 Higashi, Tsukuba, Ibaraki, Japan) on Oct. 18, 2004. That is, the Accession No. of hybridoma rSN-18 was converted to FERM BP-10143, the Accession No. of hybridoma rSN-122 was converted to FERM BP-10144, the Accession No. of hybridoma rSN-150 was converted to FERM BP-10145, the Accession No. of hybridoma rSN-21-2 was converted to FERM BP-10146, and the Accession No. of hybridoma rSN-29 was converted to FERM BP-10147, respectively.

### Example 2

### Confirmation of Reactivities of Monoclonal Antibodies by Western Blotting (WB)

The reactivity of each of the established monoclonal antibodies to the naturally occurring antigen (the N protein originated from the virus) was confirmed by WB using a concentrated virus suspension as a sample. Vero E6 cells were infected with SARS virus strain Hanoi, and the cells were cultured in a CO₂ incubator for 48 hours, followed by centrifugation of the culture medium at 2000 rpm for 15 minutes to prepare a culture supernatant (TCID₅₀ was 7.95×10⁶/ml). The culture supernatant was inactivated at 56° C. for 90 minutes, and then 31.5 ml aliquot thereof was centrifuged at 30 Krpm for 3 hours using a Hitachi ultracentrifuge (40T rotor). To the obtained precipitate, TNE (Tris-NaCl-EDTA) buffer (0.3 ml) was added, and pipetting was performed to prepare a concentrated virus suspension. To this suspension, an equivolume of sample-treating solution for electrophoresis was added, and the resulting mixture was heated to obtain a test sample. After conducting SDS-polyacrylamide gel electrophoresis (SDS-PAGE) using a 12.5% gel, the sample was transferred to a nitrocellulose membrane to prepare a transferred membrane for (WB) (antigen-transferred WB membrane). After blocking the transferred membrane with skim milk, the membrane was subjected to reaction with each of the antibodies. WB was performed using as the anti-N protein monoclonal antibodies, rSN-18 antibody, rSN-122 antibody, rSN-150 antibody, rSN-29 antibody, rSN-21-2 antibody and rSN-122 antibody, and using as a negative control, an unrelated monoclonal antibody E2CT-38 antibody.

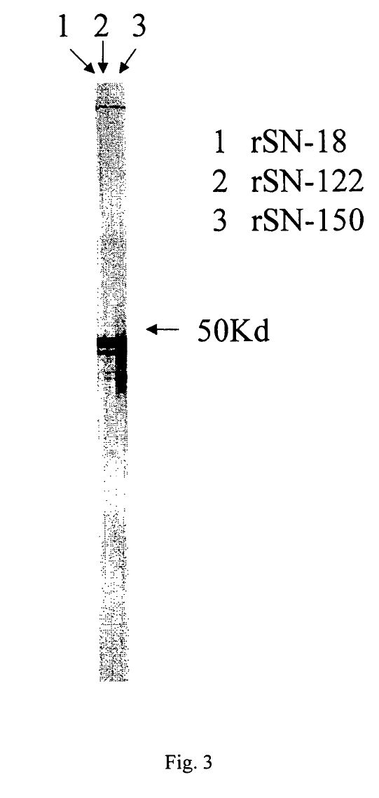

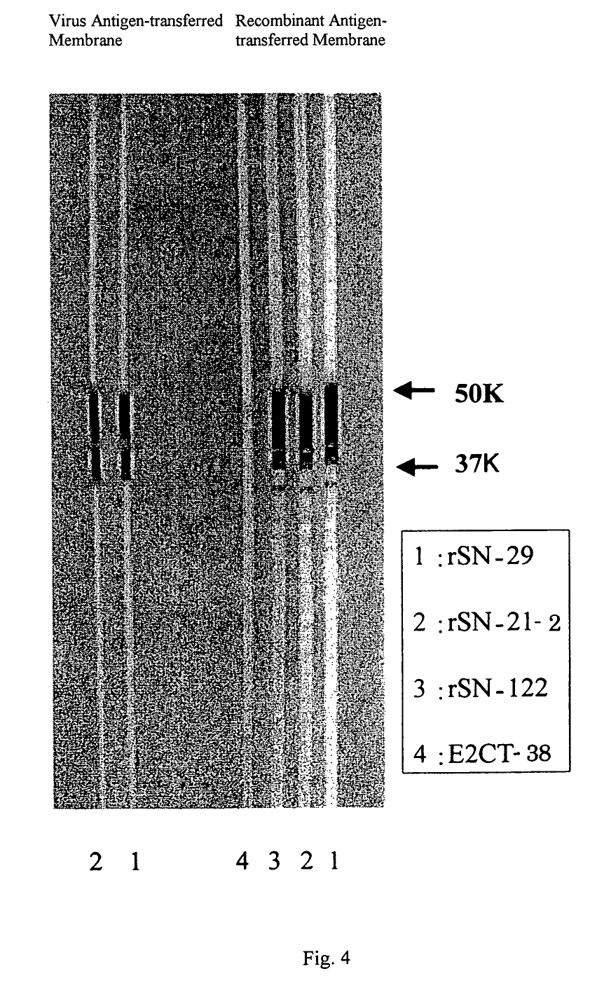

The reaction with the antibody was performed as follows: That is, each monoclonal antibody was shaken with the antigen-transferred WB membrane at room temperature for 1 hour, thereby allowing the reaction, and the membrane was washed (washing under shaking for 5 minutes) with a washing buffer (PBS containing 0.05% Tween 20). Then a POD-labeled anti-mouse immunoglobulin antibody (produced by DAKO) was added, and the reaction was carried out for another 1 hour at room temperature. After washing 4 times (washing under shaking for 5 minutes) with the washing buffer, a substrate 4-chloronaphthol solution was added, and the bands were observed. As shown in FIGS. 3 and 4, a band at a position of a molecular weight of little less than 50 Kd corresponding to the N protein was observed when each of the monoclonal antibodies was used.

### Example 3

### Detection of N Protein in Virus Culture Supernatant by Sandwich ELISA

Whether an assay system for assaying the N protein may be attained or not was tested by carrying out sandwich ELISA using the recombinant N protein and virus culture supernatant. The ELISA was carried out as follows: That is, each monoclonal antibody was diluted with PBS (pH 7.4) to a concentration of 5 μg/ml, and the antibody solution was added to each well of an ELISA plate produced by FALCON in an amount of 50 μl per well, followed by leaving the ELISA plate to stand at 4° C. overnight to coat the well with the antibody. Then 150 μl/well of 1% BSA-PBS (pH 7.4) was added to each well, and the plate was left to stand at 37° C. for 1 hour to carry out masking. Each well was washed 3 times with a washing buffer (PBS containing 0.05% Tween 20 (pH 7.4)), and then the recombinant N protein or the virus culture supernatant was added to each well in an amount of 50 μl/well, followed by allowing reaction at 37° C. for 1 hour. The recombinant N protein was used at a concentration of 20 ng/ml, and the culture supernatant was used as it is or after dilution with the washing buffer. The culture supernatant of the cells not infected with the virus was used as a negative control. Then each monoclonal antibody from each hybridoma culture supernatant was purified by using an anti-mouse immunoglobulin affinity column and pooled, followed by labeling of the monoclonal antibody with alkaline phosphatase. The obtained labeled antibody was added to each well in an amount of 50 μl/well, and reaction was allowed to occur at 37° C. for 1 hour. After washing each well 3 times with the washing buffer, the substrate p-nitrophenyl phosphate (p-NPP) was added in an amount of 50 μl/well, and the resulting mixture was left to stand at room temperature for 15 minutes. The wells were visually observed and absorbance at a wavelength of 405 nm was measured. As shown in Table 1, it was confirmed that detection of N protein may be attained with any of the monoclonal antibodies used in this Example.

TABLE 1

| | Sandwich ELISA | | Sandwich ELISA | |
|---|---|---|---|---|
| | Visual Observation | | A405 | |
| | Virus | | Virus | |
| | Culture | Control Culture | Culture | Recombinant |
| Antibody | Supernatant | Supernatant | Supernatant* | N Protein |
| rSN-18 | + | − | 0.62 | 0.46 |
| rSN-122 | + | − | 0.80 | 0.99 |
| rSN-150 | + | − | 0.90 | 1.24 |
| E2CT-38 | − | − | 0.05 | 0.10 |

*used after 4-fold dilution

### Example 4

### Preparation of Alkaline Phosphatase-Labeled Anti-SARS Virus Monoclonal Antibody

Each of the anti-SARS virus monoclonal antibodies prepared in Example 1 was reacted with 2-iminothiolane hydrochloric acid salt (produced by ALDRICH), thereby introducing thiol groups to the monoclonal antibody.

Then alkaline phosphatase to which maleimide groups were introduced and each of the above-described antibodies to which thiol groups were introduced were reacted, and the product was subjected to gel filtration to obtain purified alkaline phosphatase-labeled anti-SARS virus monoclonal antibodies.

### Example 5

### Measurement by Sandwich ELISA using Alkaline Phosphatase-Labeled Anti-SARS Virus Monoclonal Antibody

The following sandwich ELISA was performed using the recombinant N protein and inactivated virus culture supernatant obtained by heating the culture supernatant at 56° C. for 90 minutes.

Each monoclonal antibody alone or a mixture thereof was diluted to a concentration of 10 to 15 μg/ml with phosphate buffer (pH 7.5), and was placed in the wells of an IMMUNOMODULE MAXISORP plate produced by NUNC in an amount of 100 μl/well, followed by leaving the plate to stand overnight at 4° C. to immobilize the antibody. Each well was then washed 3 times with a washing buffer (TBS (Tris-buffered physiological saline) containing 0.02% Triton X-100, pH 7.2), and 1% BSA-phosphate buffer (pH 7.4) was placed in each well in an amount of 250 μl/well. The resulting plate was left to stand overnight at 37° C. to carry out blocking, thereby obtaining an antibody-immobilized plate. After washing the antibody-immobilized plate 3 times, the recombinant N protein (1.0 ng/ml) or the virus culture supernatant (100 μl/well) diluted with a reaction solution (PBS containing 1% BSA, pH 7.5) was placed in each well, and the resulting mixture was allowed to react at room temperature (25° C.) for 1 hour. A culture supernatant of the cells not infected with the virus was used as a negative control. After washing the plate 4 times with the washing buffer, the labeled antibody alone or a mixture of antibodies prepared in Example 4, at a concentration of 1.0 to 5.0 μg/ml was placed in each well in an amount of 100 μl/well, followed by allowing the mixture to react at room temperature (25° C.) for 1 hour. After washing the plate 4 times with the washing buffer, a substrate p-nitrophenyl phosphate (p-NPP) was placed in each well in an amount of 100 μl/well, and the resulting mixture was left to stand at room temperature for 30 to 60 minutes, followed by measurement of the absorbance at a wavelength of 405 nm. The results of the measurements of the absorbance obtained for the recombinant N protein and the virus culture supernatant are shown in Tables 2a and 2b, respectively. As shown in Table 2a, it was confirmed that detection of the recombinant N protein may be attained by any of the monoclonal antibodies, although the reactivities varies depending on the combination of the antibodies. As shown in Table 2b, the reactivities substantially the same as those for the recombinant N protein were observed for the virus culture supernatant.

TABLE 2a

| Labeled | Immobilized Antibody | | | | |
|---|---|---|---|---|---|
| Antibody | rSN-122 | rSN-150 | rSN-18 | rSN-21-2 | rSN-29 |
| rSN-122 | 0.029 | 0.416 | 0.253 | 0.429 | 0.439 |
| rSN-150 | 0.231 | 0.078 | 0.121 | 0.137 | 0.127 |
| rSN-18 | 0.140 | 0.136 | 0.071 | 0.067 | 0.101 |
| rSN-21-2 | 0.255 | 0.162 | 0.127 | 0.042 | 0.052 |
| rSN-29 | 0.240 | 0.140 | 0.117 | 0.028 | 0.027 |

TABLE 2b

| Labeled | Immobilized Antibody | | | | |
|---|---|---|---|---|---|
| Antibody | rSN-122 | rSN-150 | rSN-18 | rSN-21-2 | rSN-29 |
| rSN-122 | 0.069 | 2.339 | 0.197 | 1.697 | 2.264 |
| rSN-150 | 1.801 | 0.032 | 0.086 | 0.916 | 1.099 |
| rSN-18 | 0.067 | 0.080 | 0.030 | 0.049 | 0.059 |
| rSN-21-2 | 1.907 | 1.194 | 0.076 | 0.062 | 0.043 |
| rSN-29 | 2.104 | 1.260 | 0.084 | 0.040 | 0.030 |

### Example 6

### Measurement by Immunochromatography

Rapid detection of the N protein by immunochromatography using the recombinant N protein or the virus culture supernatant inactivated by heat treatment at 56° C. for 90 minutes was confirmed. An immunoassay device 1 for immunochromatography, shown in FIG. 5 was prepared as follows:

On one end of a nitrocellulose membrane 2 (5 mm×50 mm), a developer-supplying zone 3 having a substrate zone 7 prepared by spotting a 20 mg/ml solution of sodium 5-bromo-4-chloro-3-indolyl phosphate (BCIP.Na) as a substrate on a water-absorptive non-woven fabric and dried, was formed, and a water-absorptive absorption pad (developer-absorption zone 5) was formed on the other end of the membrane. A detection zone 6 was formed at a region downstream, in the direction of the liquid transportation, of the labeled reagent zone 4 (sample-spotting zone 8) in the membrane part. The detection zone 6 was prepared by spotting a solution of the monoclonal antibody (1 mg/ml) shown in Table 3a or 3b in the form of a line and drying the solution. The labeled reagent zone 4 was prepared by spotting a solution of a single or two types of the alkaline phosphatase-labeled monoclonal antibody (35 ng/pad) shown in Table 3a or 3b on a water-absorptive non-woven fabric, and drying the solution. The labeled reagent zone 4 was then mounted on the nitrocellulose membrane in which the detection zone 6 was formed, after blocking with PBS containing BSA or without the blocking.

A sample 9 (25 to 30 μl) prepared by diluting the recombinant N protein or the culture supernatant with Tris-buffered physiological saline containing 3% BSA (sample treatment solution) was spotted on the sample-spotting zone 8 formed on the labeled reagent zone 4, of the thus prepared immunoassay device 1. Then 300 μl of a developer 10 was dropped on the developer-supply zone 3 and was allowed to develop in the nitrocellulose membrane, and 15 minutes later, emergence of a line at the detection zone 6 was checked. The results are shown in Table 3a. As shown in Table 3a, although the reactivities varied depending on the combination of the antibodies, the recombinant N protein was able to be detected in a reaction time of 15 minutes. On the other hand, based on the results shown in Tables 2a, 2b and Table 3a, combinations of the immobilized antibody and the labeled antibody were selected, which showed high reactivities. Using the selected combinations, assay of the virus culture supernatant was carried out. As a result, the N protein in the virus culture supernatant was able to be detected at high dilution factors. The results are shown in Table 3b.

TABLE 3a

| Immobilized | Labeled Antibody | | |
|---|---|---|---|
| Antibody | rSN-150 | rSN-122 | rSN-18 |
| rSN-150 | — | 4w | 2 |
| rSN-122 | 3 | 3w | 4 |
| rSN-18 | 2 | 4 | — |

Values (color intensity) in the table were determined by visual observation of the color intensity of the detection line at 15 minutes from the beginning of the reaction. (4>4w>3>3w>2>2w>1, -: line not detected)

TABLE 3b

| | Immobilized Antibody | | | |
|---|---|---|---|---|
| Labeled Antibody | rsN-150 | rsN-122 | rSN-21-2 | rSN-29 |
| rSN-122 | 1500 | — | 20000 | 15000 |
| rSN-150 | — | 1500 | 1500 | 1500 |
| rSN-21-2 | 1500 | 30000 | — | — |
| rSN-29 | 3000 | 30000 | — | — |
| rSN-122 + rSN-150 | 1000 | — | >3000 | >3000 |
| rSN-122 + rSN-18 | 1000 | — | >3000 | >3000 |

The values indicate dilution factors at which the culture supernatant was able to be detected.
- indicates not determined.
>3000 indicates that a dilution factor of 3000-fold or more was able to be detected.

### Reference Example 4

### Synthesis of N5 Peptide and Preparation of KLH Conjugate

A peptide sequence (N5 peptide, GQTVTKKSAAEASKKPRC: SEQ ID NO:3) containing the amino acids 244-260 of the SARS nucleoprotein was synthesized by the Fmoc method with a peptide synthesizer produced by SHIMADZU CORPORATION (PSSM-8). N5 peptide was synthesized by the method described in the instruction of the synthesizer. The synthesized peptide was conjugated to keyhole limpet hemocyanin (KLH) to obtain a KLH conjugate.

### Example 7

### Establishment of Anti-N Protein Monoclonal Antibody using N5 Peptide Antigen

A hybridoma producing a monoclonal antibody against the N protein was prepared by immunizing a mouse with the N5 peptide-KLH conjugate prepared in Reference Example 4, and fusing lymphocytes recovered from the spleen of the mouse and myeloma cells. The details of the preparation method are described in Example 1. After screening, a hybridoma SN5-25 producing an anti-N protein monoclonal antibody was established. The monoclonal antibody obtained from this hybridoma was named SN5-25.

### Example 8

### Assay by Sandwich ELISA using Alkaline Phosphatase-Labeled Anti-SARS Virus Monoclonal Antibody

As in Example 4, alkaline phosphatase-labeled anti-SARS virus monoclonal antibodies shown in Table 4 were prepared. Further, as in Example 5, antigen-immobilized plates shown in Table 4 were prepared, and assays were performed using the virus culture supernatant. The results are shown in Table 4. As a result, the N protein in the virus culture supernatant was able to be detected with high sensitivity (samples of high dilution factor) using the monoclonal antibodies whose antigen is the peptide corresponding to the SARS nucleoprotein (244-260).

TABLE 4

| Culture Supernatant | Immobilized | Labeled | |
|---|---|---|---|
| (TCID₅₀/mL) | Antibody | Antibody | Detection |
| 3.55 × 10⁴ | SN5-25 | rSN-18 | + |
| 1.77 × 10⁴ | SN5-25 | rSN-18 | + |
| 1.22 × 10⁴ | SN5-25 | rSN-18 | + |
| | rSN-150 | rSN-122 | + |
| 8.11 × 10³ | rSN-150 | rSN-122 | + |

### INDUSTRIAL AVAILABILITY

The monoclonal antibody or the antigen-binding fragment thereof according to the present invention may be used for immunoassays for detecting or quantifying SARS virus in a test sample, and for the reagent and immunoassay device therefor.

### Sequence Listing Free Text

SEQ ID NO:3: peptide sequence consisting of amino acids 244-260 of SEQ ID NO:2 and a cystein

1. An anti-SARS virus monoclonal antibody against nucleoprotein of a corona virus which causes severe acute respiratory syndrome (SARS), or an antigen-binding fragment thereof.

2. The anti-SARS virus monoclonal antibody or the antigen-binding fragment thereof according to claim 1, which is a monoclonal antibody.

3. The anti-SARS virus monoclonal antibody or the antigen-binding fragment thereof according to claim 1, which monoclonal antibody is produced by a hybridoma prepared by using as an immunogen the nucleoprotein of said coronavirus, said nucleoprotein being expressed by a vector in which a nucleotide sequence shown in SEQ ID NO:1 is incorporated.

4. The anti-SARS virus monoclonal antibody or the antigen-binding fragment thereof according to claim 3, which monoclonal antibody has binding specificity of the monoclonal antibody produced by hybridoma rSN-18 having an Accession No. FERM BP-10143, hybridoma rSN-122 having an Accession No. FERM BP-10144, hybridoma rSN-150 having an Accession No. FERM BP-10145, hybridoma rSN-21-2 having an Accession No. FERM BP-10146 or hybridoma rSN-29 having an Accession No. FERM BP-10147.

5. The anti-SARS virus monoclonal antibody or the antigen-binding fragment thereof according to claim 1, which monoclonal antibody is produced by a hybridoma prepared by using as an immunogen the amino acid sequence shown in SEQ ID NO:3.

6. A hybridoma producing said monoclonal antibody according to claim 1, which hybridoma is obtained by fusing an anti-SARS virus monoclonal antibody-producing cell and a tumor cell.

7. Hybridoma rSN-18 having an Accession No. FERM BP-10143, hybridoma rSN-122 having an Accession No. FERM BP-10144, hybridoma rSN-150 having an Accession No. FERM BP-10145, hybridoma rSN-21-2 having an Accession No. FERM BP-10146 or hybridoma rSN-29 having an Accession No. FERM BP-10147, which hybridomas produce said monoclonal antibody or the antigen-binding fragment thereof recited in claim 1.

8. An reagent for immunoassay of SARS-causing coronavirus, comprising said monoclonal antibody or the antigen-binding fragment thereof according to claim 1 as at least one of immobilized antibody and labeled antibody.

9. An immunoassay device comprising a detection zone having an anti-SARS virus antibody immobilized on a matrix through which liquid can be transported; and a labeled reagent zone on which a labeled anti-SARS antibody is spotted in such a manner that said labeled anti-SARS antibody is mobile; at least one of said antibody immobilized on said detection zone and said labeled anti-SARS virus antibody being said monoclonal antibody or the antigen-binding fragment thereof according to claim 1.

10. The immunoassay device according to claim 9, wherein said label is an enzyme and wherein said immunoassay device has a substrate at a region upstream of said labeled reagent zone in said matrix, said substrate reacting said enzyme.

11. An immunoassay of SARS virus, comprising detecting said SARS virus in a test sample by an immunoassay utilizing antigen-antibody reaction between said anti-SARS virus monoclonal antibody or the antigen-binding fragment thereof according to claim 1 and said SARS virus in said test sample.
